(19) United States
(12) Patent Application Publication
(10) Pub. No.: US 2004/0235169 A1
(43) Pub. Date: Nov. 25, 2004

(54) Inducible expression of transfected genes

(76) Inventors: Ronald M. Evans, La Jolla, CA (US); Enrique Saez, San Diego, CA (US); Inder M Verma, La Jolla, CA (US); Francesco Galimi, San Diego, CA (US)

Correspondence Address: FOLEY & LARDNER, P.O. BOX 80278, SAN DIEGO, CA, 92138-0278, US

(21) Appl. No.: 10/475,541

(22) PCT Filed: Apr. 17, 2002
(86) PCT No.: PCT/US02/12212

Related U.S. Application Data
(60) Provisional application No. 60/285,307, filed on Apr. 20, 2001.

(51) Int. Cl. C12N 15/85

(52) U.S. Cl. 435/455; 435/320.1

(57) ABSTRACT

The present invention provides inducible gene transfer systems and gene transfer vectors for the safe and effective transfer and expression of genes in mammalian cells, and for a very high level of control of expression of the transferred genes. The inducible gene transfer systems of the present invention may be lentiviral vectors comprising a self-inactivating 5′ LTR, a modulator-responsive promoter, a nuclear import signal, a promoter operatively associated with a nucleic acid encoding a modulator-responsive receptor, an RNA stabilizing element, and a self-inactivating 3′ LTR. Thus, the present invention provides vectors for packaging and delivering DNA to both dividing and non-dividing cells. The present invention also provides methods for treating subjects with the gene transfer systems of the present invention, and cells containing the gene transfer systems.

### FIELD OF THE INVENTION

The present invention relates generally to inducible gene transfer vectors and inducible expression systems.

### BACKGROUND OF THE INVENTION

It is frequently desirable to transfer and control the expression of genes to cells or living organisms, whether the subject is cells in culture, or a living organism such as an animal model or human patient in need of receiving a therapeutic gene. When lentiviral vectors based on HIV are used as the mode of transferring and expressing genes, concerns arise regarding the safety of their use, since the virus is the etiological agent for AIDS. Further concerns involve the possibility of insertional activation of cellular oncogenes, the ability of the vector to successfully and effectively associate with ribosomes, and the ability of the vector to successfully signal for nuclear importation. To date, there has not been created a lentiviral vector that is safe and effective for use in transferring and expressing genes in mammalian hosts or cells, and which provides the important ability to control expression of the transferred genes.

### SUMMARY OF THE INVENTION

The present invention provides a solution to these problems by combining a gene transfer vector or other expression system and a gene regulation system for the efficient delivery and controlled expression of genes to cells and living organisms. The present invention therefore provides for the efficient transfection of the host, for example through the highly efficient lentivector delivery system, and for the exquisite control of the timing and level of expression of the transferred gene by the simple administration of a modulator (e.g., a steroid) to the host harboring the transferred gene. The present invention offers the additional benefit of achieving this efficient transfection and regulation in non-dividing cells in hosts of several species, such as rodents, primates, and canines.

The present invention provides inducible gene transfer vectors and expression systems. The gene transfer vectors and expression systems of the present invention may be lentiviral vectors. These vectors comprise various components that make them both safe and effective for transferring genes to mammalian host cells, and further provide the extremely important ability to exercise great control over the expression of the transferred genes in the mammalian host cells by administration of a suitable modulator to cells containing invention vectors. The lentiviral vectors of the present invention may comprise a self-inactivating 5′ LTR, a modulator-responsive promoter, a nuclear import signal, a promoter operatively associated with a nucleic acid encoding a modulator-responsive receptor, an RNA stabilizing element, and a self-inactivating 3′ LTR. Invention vectors are useful for packaging and delivering DNA to both dividing and non-dividing cells.

The present invention also provides specific vectors and methods for using the vectors to inducibly express genes of interest in target cells. The present invention further provides ex vivo methods employing invention gene transfer vectors as expression systems for treating mammalian subjects. Also provided are methods of making an animal model of expression of a gene of interest. Furthermore, the present invention provides cells incorporating or containing the gene transfer vectors or expression systems of the present invention. The present invention thus facilitates the construction of stable, inducible cell lines, as the pseudotype lentivectors can transduce many cell types that are refractory to standard DNA transfection techniques.

The present invention therefore successfully combines an efficient gene delivery system with a tightly regulated gene expression system, and represents a significant advance in gene delivery and expression technology.

### BRIEF DESCRIPTION OF THE DRAWINGS

FIG. 1 provides schematic representations of some preferred constructs of the present invention, the ecdysone-inducible lentivectors.

FIG. 2 provides a schematic representation of a preferred construct of the present invention, an ecdysone-regulated lentivector after integration.

FIG. 3 graphically depicts the expression of an ecdysone-regulated Yellow Fluorescent Protein (YFP) expression system in lentivector-infected prostate cancer cells.

FIG. 4 graphically depicts ecdysone-regulated Green Fluorescent Protein (GFP) expression in cells derived from infected human hematopoietic stem cells transplanted into mice.

FIG. 5 provides a schematic representation of lentivectors for an ecdysteroid regulatable system.

### DETAILED DESCRIPTION OF THE PREFERRED EMBODIMENTS

The present invention provides methods for regulatable gene transfer and constructs useful therefor. Thus, in accordance with one aspect of the present invention, there are provided lentivector gene delivery constructs with a modulator-responsive gene regulation system for the efficient delivery to, and controlled expression of, genes in cells and living organisms. In one embodiment of the invention, regulation of the lentivector system may be achieved by incorporating two expression cassettes in a single vector, including a transactivating cassette and an inducible expression cassette containing a reporter gene (e.g., Green Fluorescent Protein or Factor IX) or another gene of interest. In an alternate embodiment of the invention, regulation may also be achieved by co-infection strategies using two separate lentivectors, one containing the transactivating cassette and the other containing the inducible expression cassette. Either format may provide directed expression of multiple response genes in multiple cell types.

An inducible gene transfer vector of the present invention is comprised of a self-inactivating lentiviral 5′ LTR, a responsive promoter, a nuclear import signal, a promoter operatively associated with nucleic acid encoding a modulator-responsive receptor, an RNA stabilization element, and a self-inactivating 3′ LTR. Self-inactivating 5′ LTRs are described in the literature; see, for example, that described by Miyoshi et al., which will function in the present invention. Miyoshi et al., Journal of Virology, Vol. 72, No. 10 (October 1998).

In a preferred embodiment, the promotor may be modulator-responsive. The modulator-responsive promoter may comprise a binding element for a transcription factor. In other embodiments, the promoter may be responsive to hormone or hormone-like compounds. For example, the binding element may be a hormone response element. Hormone response elements typically comprise a direct or inverted repeat motif based on the half site RGBNNM, where R may be selected from A or G; B may be selected from G, C, or T; each N may be independently selected from A, T, C, or G; M may be selected from A or C; with the proviso that at least 4 nucleotides of the RGBNNM sequence are identical with the nucleotides at corresponding positions of the sequence AGTTCA; the half sites may be separated by in the range of 0 up to 15 nucleotides. In a particularly preferred embodiment, the half sites are separated by in the range of 2 up to 6 nucleotides. In another embodiment, the binding element may be a GAL4 response element, a Tet operon, and the like.

The modulator-responsive promoter may be operatively associated with a gene of interest. As readily recognized by those of ordinary skill in the art, the gene of interest can be any of a number of sequences. In various embodiments, the gene of interest may be a therapeutic gene, a reporter gene, a marker gene, a toxic gene, a regulatory gene, an enzyme, an antisense gene, or any sequence which imparts measurable properties on infected cells containing same. The gene of interest may encode a therapeutic protein, a reporter protein, a marker protein, a toxic protein, a regulatory protein, an enzyme, a ribozyme, or any sequence which imparts measurable properties on infected cells containing same.

As used herein, the phrase “operatively associated with” refers to the functional relationship of DNA with regulatory and effector sequences of nucleotides, such as promoters, enhancers, transcriptional and translational stop sites, and other signal sequences. For example, operative linkage of DNA to a promoter refers to the physical and functional relationship between the DNA and promoter such that the transcription of such DNA is initiated from the promoter by an RNA polymerase that specifically recognizes, binds to and transcribes the DNA.

The inducible gene transfer vectors of the present invention may also comprise a nuclear import signal. As readily recognized by those of ordinary skill in the art, a variety of nuclear import signals can be employed in the practice of the present invention. For example, the nuclear import signal may be central polypurine tract and termination sequences of Pol1 (cPPT), as well as additional such signals as described, for example, by Morris M C, Chaloin L, Heitz F, Divita G., “Translocating peptides and proteins and their use for gene delivery,”. Curr Opin Biotechnol. 2000 October;11(5):461-6; Jans D A, Xiao C Y, Lam M H., “Nuclear targeting signal recognition: a key control point in nuclear transport?,” Bioessays. 2000 June;22(6):532-44; Christophe D, Christophe-Hobertus C, Pichon B., “Nuclear targeting of proteins: how many different signals?,” Cell Signal. 2000 May;12(5):337-41; Luo D, Saltzman W M., “Synthetic DNA delivery systems,” Nat Biotechnol. 2000 January;18(1):33-7; Moroianu J., “Nuclear import and export pathways,” J Cell Biochem 1999 Suppl 32-33:76-83, and the like.

The inducible gene transfer vectors of the present invention may also comprise a promoter that is operatively associated with nucleic acid encoding a modulator-responsive receptor. The promoter may be constitutively active or inducible. In various embodiments, the promoter may, for example, be responsive to one or more ecdysteroids, diacyl hydrazines, xenobiotics, antibiotics, herbal extracts, prescription drugs, or the like. The person of ordinary skill in the art will realize that when the promoter is constitutively active, it may comprise a variety of promoters such as viral promoters (e.g., cytomegalovirus promoter), a cellular promoter (e.g., housekeeping genes such as β-actin or EF1α), or a tissue specific promoter (e.g., viral or cellular promoters), and the like. The person of ordinary skill in the art will also realize that when the promoter is inducible it may comprise a viral promoter (e.g., MMTV) or a cellular promoter (e.g., heat shock protein, metallothionein promoter), and the like.

The modulator-responsive receptor may be a member of the nuclear receptor superfamily. The modulator-responsive receptor may comprise an intact nuclear receptor, or may be a hybrid receptor comprising any DNA binding domain and a ligand binding domain from a member of the nuclear receptor superfamily. The person of ordinary skill in the art will readily recognize that when the modulator-responsive receptor is a member of the nuclear receptor superfamily, it may comprise any of a variety of receptors, including but not limited to the following, which are provided by way of example only: a benzoate X receptor (BXR), a constitutively active receptor (CAR), an ecdysone receptor (EcR), an estrogen receptor (ER), a glucocorticoid receptor (GR), a peroxisome proliferator activated receptor (PPAR), a progesterone receptor (PR), a pregnane X receptor (PXR), a retinoic acid receptor (RAR), a retinoid X receptor (RXR), a steroid xenobiotic receptor (SXR), a thyroid hormone receptor (TR), a vitamin D receptor (VDR), farnesoid X receptor (FXR), and the like. In presently preferred embodiments, the member of the nuclear receptor superfamily may be ecdysone receptor or a steroid xenobiotic receptor.

DNA-binding domains contemplated for use in the preparation of invention modulator-responsive receptors are typically obtained from DNA-binding proteins (e.g., transcription factors). The term “DNA-binding domain” is understood in the art to refer to an amino acid sequence that is able to bind to DNA. As used herein, the term “DNA-binding domain” encompasses a minimal peptide sequence of a DNA-binding protein, up to the entire length of a DNA-binding protein, so long as the DNA-binding domain functions to associate with a particular response element.

Such DNA-binding domains are known to function heterologously in combination with other functional protein domains by maintaining the ability to bind the natural DNA recognition sequence (see, e.g., Brent and Ptashne, 1985, Cell, 43:729-736). For example, hormone receptors are known to have interchangeable DNA-binding domains that function in chimeric proteins (see, e.g., U.S. Pat. No. 4,981,784; and Evans, R., 1988, Science, 240:889-895). Thus, similar to the ligand binding domain of invention modified ecdysone receptor, the DNA-binding domain can be positioned at either the carboxy terminus or the amino terminus, or the DNA-binding domain can be positioned between the ligand binding domain and the activation domain. In preferred embodiments of the present invention, the DNA-binding domain is positioned internally between the ligand binding domain and the activation domain.

“DNA-binding protein(s)” contemplated for use herein belong to the well-known class of proteins that are able to directly bind DNA and facilitate initiation or repression of transcription. Exemplary DNA-binding proteins contemplated for use herein include transcription control proteins (e.g., transcription factors and the like, such as those described by Conaway and Conaway, 1994, “Transcription Mechanisms and Regulation”, Raven Press Series on Molecular and Cellular Biology, Vol. 3, Raven Press, Ltd., New York, N.Y.).

Transcription factors contemplated for use herein as a source of such DNA binding domains include, e.g., homeobox proteins, zinc finger proteins, hormone receptors, helix-turn-helix proteins, helix-loop-helix proteins, basic-Zip proteins (bZip), β-ribbon factors, and the like. See, for example, Harrison, S., “A Structural Taxonomy of DNA-binding Domains,” Nature, 353:715-719. Homeobox DNA-binding proteins suitable for use herein include, for example, HOX, STF-1 (Leonard et al., 1993, Mol. Endo., 7:1275-1283), Antp, Mat α-2, INV, and the like. See, also, Scott et al. (1989), Biochem. Biophys. Acta, 989:25-48. It has been found that a fragment of 76 amino acids (corresponding to amino acids 140-215 described in Leonard et al., 1993, Mol. Endo., 7:1275-1283) containing the STF-1 homeodomain binds DNA as tightly as wild-type STF-1. Suitable zinc finger DNA-binding proteins for use herein include Zif268, GLI, XFin, and the like. See also, Klug and Rhodes (1987), Trends Biochem. Sci., 12:464; Jacobs and Michaels (1990), New Biol., 2:583; and Jacobs (1992), EMBO J., 11:4507-4517.

Preferably, the DNA-binding domain used herein is obtained from a member of the steroid/thyroid hormone superfamily of receptors. As used herein, the phrase “member(s) of the steroid/thyroid hormone superfamily of receptors” (also known as “nuclear receptors” or “intracellular receptors”) refers to hormone binding proteins that operate as ligand-dependent transcription factors, including identified members of the steroid/thyroid hormone superfamily of receptors for which specific ligands have not yet been identified (referred to hereinafter as “orphan receptors”).

Exemplary members of the steroid/thyroid hormone superfamily of receptors (including the various isoforms thereof) include steroid receptors such as glucocorticoid receptor (GR), mineralocorticoid receptor (MR), estrogen receptor (ER), progesterone receptor (PR), androgen receptor (AR), vitamin D₃ receptor (VDR), and the like; plus retinoid receptors, such as the various isoforms of retinoic acid receptor (e.g., RARα, RARβ, or RARγ), the various isoforms of retinoid X receptor (e.g., RXRα, RXRβ, or RXRγ), and the like (see, e.g., U.S. Pat. Nos. 4,981,784; 5,171,671; and 5,071,773); thyroid receptors (TR), such as TRα, TRβ, and the like; insect derived receptors such as the ecdysone receptor, and the like; as well as other gene products which, by their structure and properties, are considered to be members of the superfamily, as defined hereinabove, including the various isoforms thereof. Examples of orphan receptors contemplated for use herein as a source of DNA binding domain include HNF4 (see, for example, Sladek et al., in Genes & Development 4: 2353-2365 (1990)), the COUP family of receptors (see, for example, Miyajima et al., in Nucleic Acids Research 16: 11057-11074 (1988), and Wang et al., in Nature 340: 163-166 (1989)), COUP-like receptors and COUP homologs, such as those described by Mlodzik et al., in Cell 60: 211-224 (1990) and Ladias et al., in Science 251: 561-565 (1991), various isoforms of peroxisome proliferator-activated receptors (PPARs; see, for example, Issemann and Green, supra), the insect derived knirps and knirps-related receptors, and the like.

The DNA-binding domains of all members of the steroid/thyroid hormone superfamily of receptors are related, consisting of 66-68 amino acid residues, and possessing about 20 invariant amino acid residues, including nine cysteines. A member of the superfamily can be characterized as a protein which contains these 20 invariant amino acid residues. The highly conserved amino acids of the DNA-binding domain of members of the superfamily are as follows:

Cys-X-X-Cys-X-X-Asp*-X-Ala*-X-Gly*-X-Tyr*-X-
X-X-X-Cys-X-X-Cys-Lys*-X-Phe-Phe-X-Arg*-X-
X-X-X-X-X-X-X-X-(X-X-)Cys-X-X-X-X-X-(X-
X-X-)Cys-X-X-X-Lys-X-X-Arg-X-X-Cys-X-X-Cys-
Arg*-X-X-Lys*-Cys-X-X-X-Gly*-Met;
(SEQ ID NO: 1)

wherein X designates non-conserved amino acids within the DNA-binding domain; an asterisk denotes the amino acid residues which are almost universally conserved, but for which variations have been found in some identified hormone receptors; and the residues enclosed in parenthesis are optional residues (thus, the DNA-binding domain is a minimum of 66 amino acids in length, but can contain several additional residues).

Modification of existing DNA-binding domains to recognize new target recognition sequences is also contemplated herein. For example, in accordance with the present invention, it has been found that the modification of the “P-box” sequence of DNA-binding domains of members of the steroid/thyroid hormone superfamily of receptors offers unique advantages not present in other chimeric hormone receptors. For example, the modification of a P-box amino acid sequence to preferentially bind to a different hormone response element half-site than the naturally occurring P-box amino acid sequence can reduce undesired background levels of gene expression. Thus, invention receptors and methods provide the advantage of increasing the selectivity of exogenous gene expression in a particular subject.

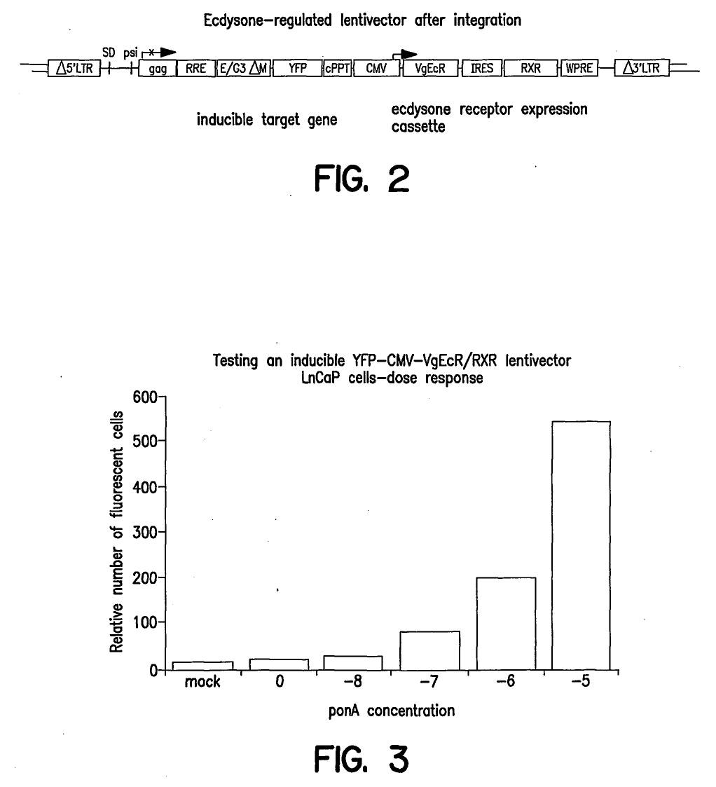

As used herein, the phrase “P-box amino acid sequence” refers to the proximal element region in a DNA-binding domain of a hormone receptor that typically occurs at the junction of the first zinc finger and the linker region, for example, at about amino acids 19-23 of the DNA-binding domain (i.e., amino acids 19-23 of SEQ ID NO:1); see, for example, Umesono et al. (1989), Cell, 57:1139-1146, FIG. 2 and Table 1, who describe various naturally occurring P-box amino acid sequences for a variety of hormone receptor DNA-binding domains.

It has also been found that in vitro evolution methods can be applied to modify and improve existing DNA-binding domains (see, e.g., Devlin et al., 1990, Science, 249:404-406; and Scott and Smith, 1990, Science, 249:386-390).

The inducible gene transfer vectors of the present invention may optionally include a silent partner for the modulator-responsive receptor. When the silent partner is present, it may comprise Retinoid X Receptor (RXR), ultraspiracle receptor (USP), or the like, or a functional fragment thereof. By functional fragment is meant at least the ligand binding domain and/or the dimerization domain thereof. When the gene transfer vector of the invention encodes both a modulator-responsive receptor and a silent partner therefor, efficient expression of both proteins is facilitated by incorporation of an internal ribosomal entry site in the construct.

The inducible gene transfer vector of the present invention may further comprise an RNA stabilization element. As readily recognized by those of ordinary skill in the art, a variety of RNA stabilization elements can be employed in the practice of the invention. For example, the stabilization element may be post-transcriptional regulatory element of woodchuck hepatitis virus (wpre), and the like.

The inducible gene transfer vector of the present invention may further comprise a self-inactivating lentiviral 3′ LTR. Self-inactivating 3′ LTRs are described in the literature; see, for example, the LTR described by Miyoshi et al. As an example of what will function in the practice of the present invention. Miyoshi et al., Journal of Virology, Vol. 72, No. 10 (October 1998).

In another aspect of the present invention, gene transfer can be accomplished employing a pair of cooperative vectors, i.e., the first member of the pair being a transactivating lentivector comprising a self-inactivating lentiviral 5′ LTR, a nuclear import signal, a promoter operatively associated with nucleic acid encoding a modulator-responsive receptor (and, optionally, a silent partner therefor), an RNA stabilization element, and a self-inactivating lentiviral 3′ LTR; and the second member of the pair being a response lentivector comprising a self-inactivating lentiviral 5′ LTR, a nuclear import signal, a modulator-responsive promoter operatively associated with a gene of interest, an RNA stabilization element, and a self-inactivating leintiviral 3′ LTR.

In yet another aspect, the present invention provides an inducible expression system comprising a combination of the above-described transactivating lentivector and response lentivector of the present invention.

In still another aspect, the present invention provides methods for treating a subject comprising introducing an inducible expression system or gene transfer vector of the present invention into said subject, and then exposing the subject to an effective amount of at least one modulator for the modulator-responsive receptor, such as those modulators described above. The invention methods may further comprise exposing the subject to a silent partner or functional fragment thereof, for the modulator-responsive receptor. Additionally, invention methods may comprise exposing the subject to a modulator for the silent partner or functional fragment thereof.

As employed herein, the terms “modulate” and “modulating” refer to the ability of a given modulator/receptor complex to effect transactivation of transcription of an exogenous gene, relative to such ability of said receptor in the absence of modulator. The actual effect of complex formation on the transactivation activity of a receptor will vary depending on the specific receptor species which are part of the modulator/receptor complex, and on the response element with which the modulator/receptor complex interacts.

As used herein, when referring to genes, the phrase “exogenous to said mammalian subject” or simply “exogenous” refers to any gene wherein the gene product is not naturally expressed in the particular cell where expression is desired. For example, exogenous genes can be either natural or synthetic wild type genes and therapeutic genes, which are introduced into the subject in the form of DNA or RNA. The gene of interest can be introduced into target cells (for in vitro applications), or the gene of interest can be introduced directly into a subject, or indirectly introduced by the transfer of transformed cells into a subject.

“Wild type” genes are those that are native to cells of a particular type. Such genes may be undesirably overexpressed, or may not be expressed in biologically significant levels. Thus, for example, while a synthetic or natural gene coding for human insulin would be exogenous genetic material to a yeast cell (since yeast cells do not naturally contain insulin genes), a human insulin gene inserted into a human skin fibroblast cell would be a wild type gene with respect to that cell since human skin fibroblasts contain genetic material encoding human insulin, although human skin fibroblasts do not express human insulin in biologically significant levels.

Wild type genes contemplated for use in the practice of the present invention include genes which encode a gene product:

the substantial absence of which leads to the occurrence of a non-normal state in said subject; or

a substantial excess of which leads to the occurrence of a non-normal state in said subject;

and the like.

As employed herein, the phrase “therapeutic gene” refers to a gene which imparts a beneficial function to the host cell in which such gene is expressed. In accordance with the methods described herein, therapeutic genes are expressed at a level that provides a therapeutically effective amount of the corresponding therapeutic protein. The effective amount of modulator contemplated for use in the practice of the present invention is the amount of modulator (e.g., ecdysteroid) required to achieve the desired level of gene expression product. Modulator can be administered in a variety of ways, as are well-known in the art. For example, such modulators can be administered topically, orally, intravenously, intraperitoneally, intravascularly, and the like.

Therapeutic genes contemplated for use in the practice of the present invention include genes which encode a gene product:

which is toxic to the cells in which it is expressed; or

which imparts a beneficial property to the host subject (e.g., disease resistance, etc);

and the like.

Numerous genomic and cDNA nucleic acid sequences coding for a variety of proteins are well known in the art. Exogenous genetic material useful in the practice of the present invention include genes that encode biologically active proteins of interest, such as, e.g., secretory proteins that can be released from said cell; enzymes that can metabolize a substrate from a toxic substance to a non-toxic substance, or from an inactive substance to a useful substance; regulatory proteins; cell surface receptors; and the like. Useful genes include genes that encode blood clotting factors such as human factors VIII and IX; genes that encode hormones such as insulin, parathyroid hormone, luteinizing hormone releasing factor (LHRH), alpha and beta seminal inhibins, and human growth hormone; genes that encode proteins such as enzymes, the absence of which leads to the occurrence of an abnormal state; genes encoding cytokines or lymphokines such as interferons, granulocytic macrophage colony stimulating factor (GM-CSF), colony stimulating factor-1 (CSF-1), tumor necrosis factor (TNF), and erythropoietin (EPO); genes encoding inhibitor substances such as alpha₁-antitrypsin; genes encoding substances that function as drugs, e.g., genes encoding the diphtheria and cholera toxins; and the like.

Typically, nucleic acid sequence information for a desired protein can be located in one of many public access databases, e.g., GENBANK, EMBL, Swiss-Prot, and PIR, or in many biology related journal publications. Thus, those of skill in the art have access to nucleic acid sequence information for virtually all known genes. Those of skill in the art can either obtain the corresponding nucleic acid molecule directly from a public depository or the institution that published the sequence. Optionally, once the nucleic acid sequence encoding a desired protein has been ascertained, the skilled artisan can employ routine methods, e.g., polymerase chain reaction (PCR) amplification, to isolate the desired nucleic acid molecule from the appropriate nucleic acid library. Thus, all known nucleic acids encoding proteins of interest are available for use in the methods and products described herein.

As used herein, the terms “mammal” and “mammalian” refer to humans; domesticated animals, e.g., rats, mice, rabbits, canines, felines, and the like; farm animals, e.g., chickens, bovine, porcine and ovine, and the like; and animals of zoological interest, e.g., monkeys and baboons, and the like.

The terms “ecdysone”, “ecdysteroid+38, “ecdysone-analogs”, and “ecdysone mimics” as interchangeably used herein, are employed herein in the generic sense (in accordance with common usage in the art), referring to a family of modulators with the appropriate binding and transactivation activity (see, for example, Cherbas et al., in Biosynthesis, metabolism and mode of action of invertebrate hormones (ed. J. Hoffmann and M. Porchet), p. 305-322; Springer-Verlag, Berlin). “Ecdysone” as used herein may therefore embrace a steroid, steroid-like or non-steroidal compound which acts to modulate gene transcription for a gene maintained under the control of a suitable response element, as described herein.

20-Hydroxy-ecdysone (also known as β-ecdysone) is the major naturally occurring ecdysone. Unsubstituted ecdysone (also known as a-ecdysone) is converted in peripheral tissues to β-ecdysone. Analogs of the naturally occurring ecdysones are also contemplated within the scope of the present invention. Examples of such analogs, commonly referred to as ecdysteroids, include ponasterone A, ponasterone B, ponasterone C, ponasterone D, 26-iodoponasterone A, muristerone A, inokosterone, 26-mesylinokosterone, sidasterone, buterosterone, ajugasterone, makisterone, cyasterone, sengosterone, and the like. Since it has been previously reported that the above-described ecdysones are neither toxic, teratogenic, nor known to affect mammalian physiology, they are ideal candidates for use as inducers in cultured cells and transgenic mammals according to the invention methods.

Additional compounds contemplated for use herein are mimics of the naturally occurring ecdysones, i.e., synthetic organic compounds which have binding and transactivation activities characteristic of the naturally occurring ecdysones. Examples of such compounds, referred to herein as ecdysone mimics, include 1,2-diacyl hydrazines (e.g., those described in U.S. Pat. Nos. 5,424,333 and 5,354,762, the entire contents of each of which are hereby incorporated by reference herein), N′-substituted-N,N′-di-substituted hydrazines (e.g., those described in U.S. Pat. No. 5,117,057, the entire contents of which are hereby incorporated by reference herein), dibenzoylalkyl cyanohydrazines (e.g., those described in European Application No. 461,809, the entire contents of which are hereby incorporated by reference herein), N-substituted-N-alkyl-N,N′-diaroyl hydrazines (e.g., those described in U.S. Pat. No. 5,225,443, the entire contents of which are hereby incorporated by reference herein), N-substituted-N-acyl-N-alkyl, carbonyl hydrazines (e.g., those described in European Application No. 234,944, the entire contents of which are hereby incorporated by reference herein), N-aroyl-N′-alkyl-N′-aroyl hydrazines (e.g., those described in U.S. Pat. No. 4,985,461, the entire contents of which are hereby incorporated by reference herein), and the like. Compounds of specific interest are those having the formula:

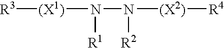

wherein:

R¹ is optionally hydrogen, lower alkyl or substituted lower alkyl, alkenyl or substituted alkenyl, alkynyl or substituted alkynyl, aryl or substituted aryl, heteroaryl or substituted heteroaryl, and the like. R¹ is not present when X¹ is part of a carbon-nitrogen double bond linking R³ to the hydrazino group;

R² is optionally hydrogen, alkyl or substituted alkyl, cyclohexyl or substituted cyclohexyl, and the like. R² is not present when X² is part of a carbon-nitrogen double bond linking R⁴ to the hydrazino group;

R³ and R⁴ are independently part of an appropriately substituted carbon-nitrogen double bond which links R³ and/or R⁴ to the hydrazino linkage, or R³ and R⁴ are independently aryl or substituted aryl, heteroaryl or substituted heteroaryl, provided, however, that when two adjacent positions on the aryl or heteroaryl moieties are substituted with alkoxy, thioalkyl, alkylamino, or dialkylamino groups, these groups may be joined to form a 5- or 6-membered heterocyclic ring system, or R³ and R⁴ are independently heterocyclic or substituted heterocyclic, cycloalkyl or substituted cycloalkyl, and the like; and

X¹ and X² are independently —C(O)—, —C(S)—, —C(NR₂)—, —C(═CN)NH—, —C(O)O—, —C(O)NH—, —C(O)NHSO₂—, —CH₂—, —SO₂—, —P(O)CH₃—, and the like, as well as an appropriate substituted carbon-nitrogen double bond which links R³ and/or R⁴ to the hydrazino linkage.

As employed herein, “alkyl” refers to alkyl groups having in the range of 1 up to 8 carbon atoms; “lower alkyl” refers to alkyl groups having in the range of 1 up to 4 carbon atoms; and “substituted alkyl” or “substituted lower alkyl” comprises alkyl (or lower alkyl) groups further bearing one or more substituents selected from halogen, cyano, nitro, hydroxy, alkoxy (—OR), thioalkyl (—SR), —NR₂, —NRC(O)R, —OC(O)R, —C(O)OR, —C(O)NR₂, —C(O)R, wherein each R is independently hydrogen or lower alkyl, and the like.

As employed herein, “cycloalkyl” refers to cyclic ring-containing groups containing in the range of about 5 up to 8 carbon atoms, and “substituted cycloalkyl” refers to cycloalkyl groups further bearing one or more substituents as set forth above, as well as lower alkyl.

As employed herein, “heterocyclic” refers to cyclic (i.e., ring-containing) groups containing one or more (up to four) heteroatoms (e.g., N, O, S, or the like) as part of the ring structure, and having in the range of 2 up to 5 nuclear carbon atoms and “substituted heterocyclic” refers to heterocyclic groups further bearing one or more substituents as set forth above, as well as lower alkyl.

As employed herein, “alkenyl” refers to straight or branched chain hydrocarbyl groups having at least one carbon-carbon double bond, and having in the range of about 2 up to 12 carbon atoms, and “substituted alkenyl” refers to alkenyl groups further bearing one or more substituents as set forth above.

As employed herein, “alkynyl” refers to straight or branched chain hydrocarbyl groups having at least one carbon-carbon triple bond, and having in the range of about 2 up to 12 carbon atoms, and “substituted alkynyl” refers to alkynyl groups further bearing one or more substituents as set forth above.

As employed herein, “aryl” refers to aromatic groups having in the range of 6 up to 14 carbon atoms and “substituted aryl” refers to aryl groups further bearing one or more substituents as set forth above, as well as lower allyl.

As employed herein, “heteroaryl” refers to aromatic groups containing one or more heteroatoms (e.g., N, O, S, or the like) as part of the ring structure, and having in the range of 3 up to 14 carbon atoms and “substituted heteroaryl” refers to heteroaryl groups further bearing one or more substituents as set forth above.

Presently preferred ecdysone mimics contemplated for use herein include compounds wherein R¹ is hydrogen; R² is an alkyl group possessing considerable bulk (such as, for example, alkyl groups containing a tertiary carbon center, e.g., —C(R″)₃, wherein each R″ is methyl or greater). Examples of alkyl groups having sufficient bulk for use herein include tert-butyl, sec-butyl, isopropyl, isobutyl, cyclohexyl, cyclopentyl, dicyclopropylmethyl, (cyclohexyl)ethyl, and the like); X¹ and X² are both —C(O)—; R³ is phenyl, substituted phenyl (with hydroxy, alkoxy, halo and/or substituted amino substituents being preferred, with 3,4-disubstitution pattern being especially preferred), heterocyclic (e.g., pyridyl or pyrimidine) or substituted heterocyclic (with halo, alkyl, thioalkyl, hydroxy, alkoxy, and/or amino substituents being preferred); and R⁴ is phenyl or substituted phenyl, heteroaryl or substituted heteroaryl or a bulky alkyl or cycloalkyl group.

Especially preferred ecdysone mimics contemplated for use herein include N′-(3,5-dimethylbenzoyl)-N-(4-ethylbenzoyl)-N′-(tert-butyl) hydrazine, N,N′-dibenzoyl-N′-(tert-butyl) hydrazine, N′-(3,5-dimethylbenzoyl)-N-(4-ethylbenzyl)-N′-(tert-butyl) hydrazine, N′-(3,5-dimethylbenzoyl)-N-(2-methyl-3,4-(ethylenedioxy)-benzoyl)-N′-(tert-butyl) hydrazine, 3,5-di-tert-butyl-4-hydroxy-N-isobutyl-benzamide, 8-O-acetylharpagide, and the like.

Modulators for the silent partner contemplated for use in the practice of the present invention is a compound which interacts (directly or indirectly) with a silent partner for the modulator receptor described herein. While such modulators alone impart virtually no activity to the invention expression system, they have been discovered to greatly enhance the ability of modulators to modulate the invention expression system. Those of skill in the art can readily determine suitable modulators for the silent partner being employed. A presently preferred silent partner is RXR; exemplary RXR agonists contemplated for use herein include 9-cis-retinoic acid, 4-(1-(3,5,5,8,8-pentamethyl-5,6,7,8-tetrahydro-2-naphthyl)-ethenyl)benzoic acid (3-methyl-TTNEB; LGD 1069), ((E)-2-(2-(5,6,7,8-tetra-hydro-3,5,5,8,8-pentamethyl-2-naphthyl)propen-1-yl)-4-thiophenecarboxylic acid) (AGN 191701), 2-(5,6,7,8-tetra-hydro-5,5,8,8-tetramethyl-2-naphthyl)-2-(carboxyphenyl)-1,3-dioxolane (SR 11237), 4-(5H-2,3-(2,5-dimethyl-2,5-hexano)-5-methyl-dibenzo(b,e)(1,4)diazepin-11-yl)-benzoic acid (HX600) or thiadiazepin analogs thereof, 3,7,11,15-tetramethyl hexadecanoic acid (phytanic acid), 6-(1-(3,5,5,8,8-pentamethyl-5,6,7,8-tetrahydronaphthalen-2-yl)cyclopropyl)nicotinic acid (LG1000268), 2-(4-carboxyphenyl)-2-(5,6,7,8-tetrahydro-5,5,8,8-tetramethyl-2-naphthalenyl)-1,3-dithiane (SR11203), 4-(2-methyl)-1-(5,6,7,8-tetrahydro-5,5,8,8-tetramethyl-2-naphthalenyl)propenyl)benzoic acid (SR11217), and the like.

In another aspect, the present invention provides methods of imparting to a target cell the ability to inducibly express a gene of interest. Invention methods may comprise introducing an expression system or gene transfer vector of the present invention into the target cells.

In yet another aspect, the present invention provides methods of inducibly expressing a gene of interest in a target cell by-infecting a target cell with an expression system or gene transfer vector of the present invention and exposing the target cell to a modulator for a modulator-responsive receptor. Invention methods may further comprise exposing the cell to a silent partner, or functional fragment thereof, for the modulator-responsive receptor. In yet another embodiment, invention methods may further include exposing the cell to a modulator for the silent partner or functional fragment thereof.

In still another aspect, the present invention provides ex vivo methods for treating a subject. These methods may include introducing an inducible expression system or gene transfer vector of the present invention into cells compatible with the subject, and introducing the modified cells into the subject. In preferred embodiments, the cells compatible with the subject are obtained from the subject, the inducible expression system or gene transfer vector is introduced into the cells, and the cells are re-introduced to the subject.

In a further aspect, the present invention provides methods of making an animal model for expressing of a gene of interest. This can be accomplished by infecting progenitor cells with an inducible expression system or gene transfer vector of the present invention, and proliferating and differentiating the infected progenitor cells into a viable animal. The animal may be a canine, mice, rabbits and other rodents, an equine, a porcine, or any mammal, including humans. In preferred embodiments, the viable animal may be an animal model produced by any of the methods of the present invention. In other embodiments, invention methods may include infecting a host animal with an inducible expression system or gene transfer is vector of the present invention.

In a still further aspect, the present invention also provides cells containing or incorporating an expression system or gene transfer vector of the present invention.

Therefore, among the many potential applications of invention methods and constructs useful therefor, the present invention may find application for transfer and regulation of therapeutic genes to patients, with regulated expression thereof, or for experimental use in cultured cells (both dividing and non-dividing) and living organisms.

Recombinant products detrimental to a host organism contemplated for expression in accordance with the present invention include any gene product that functions to confer a toxic effect on the organism. For example, inducible expression of a toxin such as the diptheroid toxin would allow for inducible tissue specific ablation (Ross et al. (1993) Genes and Development 7, 1318-1324). Thus, the numerous gene products that are known to induce apoptosis in cells expressing such products are contemplated for use herein (see, e.g, Apoptosis, The Molecular Basis of Cell Death, Current Communications In Cell & Molecular Biology, Cold Spring Harbor Laboratory Press, 1991).

The present invention therefore provides effective delivery and integration of regulatable cassettes for a variety of in vitro and in vivo applications. The invention may be used to target both dividing and non-dividing cells of many different tissue types in vitro and in vivo, therefore providing a significant advantage over other gene delivery systems. Modulators contemplated for controlling gene expression in accordance with the present invention may be small lipophilic compounds that penetrate all tissues, thus providing an additional advantage. Such modulators possess favorable pharmacokinetics of rapid clearance and uptake, and are safe and non-toxic, providing another important advantage. Modulators can be activators or inhibitors of the modulator-responsive receptors of the invention, and can act directly or indirectly, i.e., a modulator contemplated for use herein can act by direct binding to the modulator-responsive receptor, or as a precursor of a compound which so binds, or as an inducer of a cellular change which alters the activity of the modulator-responsive receptor. The gene expression regulation systems of the present invention do not interfere with endogenous cellular machinery, thereby providing for both tight control over gene expression and preventing plieotropic effects on the host cell or organism.

In accordance with a particular embodiment of the present invention, pharmaceutically acceptable formulations, and kits thereof, comprising at least one modulator, and a pharmaceutically acceptable carrier are contemplated. In accordance with another aspect of the present invention, pharmaceutically acceptable formulations consisting essentially of at least one modulator and a pharmaceutically acceptable carrier, are contemplated. Pharmaceutical formulations of the present invention can be used in the form of a solid, a solution, an emulsion, a dispersion, a micelle, a liposome, and the like, wherein the resulting formulation contains one or more of the modulators of the present invention, as an active ingredient, in admixture with an organic or inorganic carrier or excipient suitable for enteral or parenteral applications.

The active ingredient may be compounded, for example, with the usual non-toxic, pharmaceutically acceptable carriers suitable for oral, topical, nasal, transdermal, intravenous, subcutaneous, intramuscular, intracutaneous, intraperitoneally, intravascular and the like administration. Administration in the form of creams, lotions, tablets, dispersible powders, granules, syrups, elixirs, sterile aqueous or non-aqueous solutions, suspensions or emulsions, and the like, is contemplated. Exemplary pharmaceutically acceptable carriers include carriers for tablets, pellets, capsules, suppositories, solutions, emulsions, suspensions, and any other form suitable for use. Such carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. The active compound (i.e., ecdysteroid as described herein) is included in the pharmaceutically acceptable formulation in an amount sufficient to produce the desired effect upon the process or condition of diseases.

Pharmaceutically acceptable formulations containing the active ingredient may be in a form suitable for oral use, for example, as aqueous or oily suspensions, syrups or elixirs, tablets, troches, lozenges, dispersible powders or granules, emulsions, or hard or soft capsules. For the preparation of oral liquids, suitable carriers include emulsions, solutions, suspensions, syrups, and the like, optionally containing additives such as wetting agents, emulsifying and suspending agents, dispersing agents, sweetening, flavoring, coloring, preserving and perfuming agents, and the like. Formulations intended for oral use may be prepared according to any method known to the art for the manufacture of pharmaceutically acceptable formulations.

Tablets containing the active ingredient in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,160,452; and 4,265,874, to form osmotic therapeutic tablets for controlled release.

In some cases, formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, for example, peanut oil, liquid paraffin, or olive oil.

The pharmaceutically acceptable formulations may be in the form of a sterile injectable suspension. Suitable carriers include non-toxic parenterally-acceptable sterile aqueous or non-aqueous solutions, suspensions, or emulsions. This suspension may be formulated according to known methods using suitable dispersing or wetting agents and suspending agents. They can also be manufactured in the form of sterile water, or some other sterile injectable medium immediately before use. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. They may be sterilized, for example, by filtration through a bacteria-retaining filter, by incorporating sterilizing agents into the formulations, by irradiating the formulations, or by heating the formulations. Sterile injectable suspensions may also contain adjuvants such as preserving, wetting, emulsifying, and dispersing agents. Buffers, preservatives, antioxidants, and the like can be incorporated as required.

Compounds contemplated for use in the practice of the present invention may also be administered in the form of suppositories for rectal administration of the drug. These formulations may be prepared by mixing the drug with a suitable nonirritating excipient, such as cocoa butter, synthetic glyceride esters of polyethylene glycols, which are solid at ordinary temperatures, but liquefy and/or dissolve in the rectal cavity to release the drug.

The pharmaceutically acceptable formulations are administered in a manner compatible with the route of administration, the dosage formulation, and in a therapeutically effective amount. The required dosage will vary with the particular treatment desired, the degree and duration of therapeutic effect desired, the judgment of the practitioner, as well as properties peculiar to each individual. Moreover, suitable dosage ranges for systemic application depend on the route of administration. It is anticipated that dosages between about 10 micrograms and about 1 milligram per kilogram of body weight per day will be used for therapeutic treatment.

An effective amount of the pharmaceutically acceptable formulation contemplated for use in the practice of the present invention is the amount of the pharmaceutically acceptable formulation (e.g., ecdysteroids(s)) required to achieve the desired level of transcription and/or translation of exogenous nucleic acid. A therapeutically effective amount is typically an amount of a ligand or ligand precursor that, when administered in a pharamceutically acceptable formulation, is sufficient to achieve a plasma concentration of the transcribed or expressed nucleic acid product from about 0.1 μg/ml to about 100 μg/ml, preferably from about 1.0 μg/ml to about 50 μg/ml, more preferably at least about 2 μg/ml and usually 5 to 10 μg/ml.

Pharmaceutically acceptable formulations containing suitable ligand(s) are preferably administered intravenously, as by injection of a unit dose, for example. The term “unit dose,” when used in reference to a pharmaceutically acceptable formulation of the present invention, refers to a quantity of the pharmaceutical formulation suitable as unitary dosage for the subject, each unit containing a predetermined quantity of active material calculated to produce the desired therapeutic effect in association with the required diluent, i.e., carrier, or vehicle. It may be particularly advantageous to administer such formulations in depot or long-lasting form as discussed hereinafter.

Further embodiments and advantages of the present invention will be illustrated with reference to the following non-limiting examples.

### EXAMPLE 1

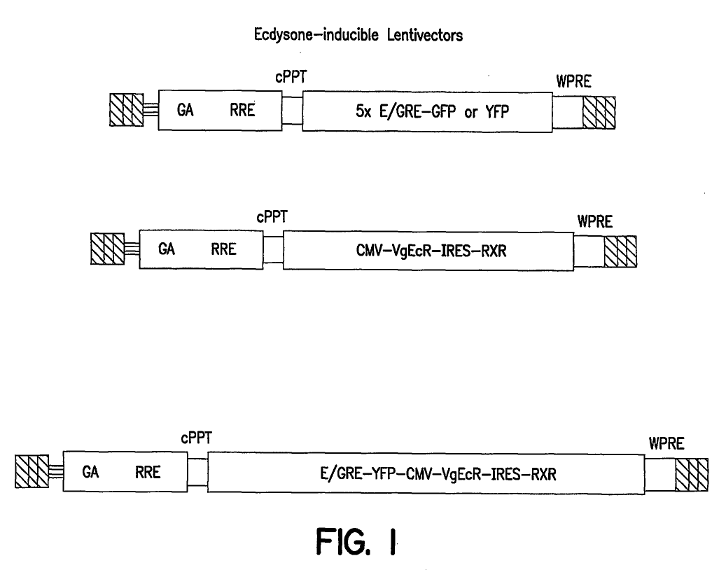

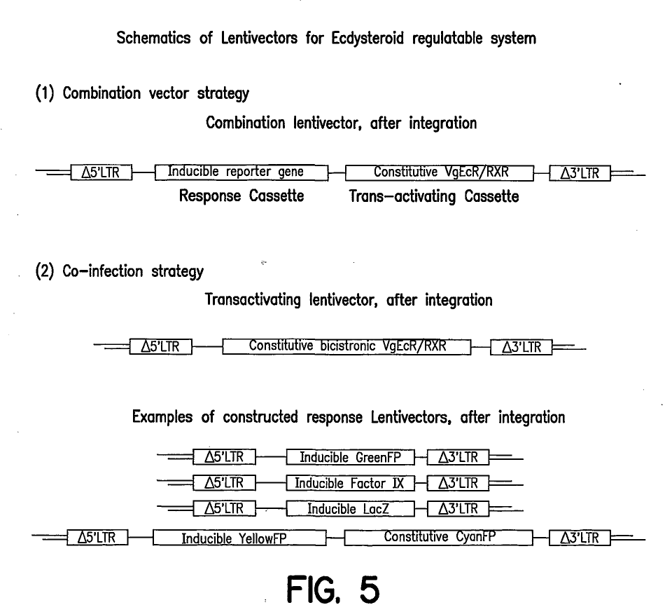

Referring to FIGS. 1 and 5, there are illustrated embodiments of the present invention. FIGS. 1 and 5 illustrate ecdysone-inducible lentivectors. In this embodiment, LnCaP cells (a prostate cancer cell line) were infected with PG-cPPT-E/GYFP-CVgRXR-WPRE, a self-inactivating lentivector that introduces a gene of interest (YFP in this embodiment) under the control of an ecdysteroid-reponsive promoter (E/G3 M), and the transactivating receptors (VgEcR and RXR) under the control a constitutive promoter (CMV). Twenty-four hours after infection various concentrations of inducer (ponasterone A) were added to the media. The number of YFP-expressing cells was measured 48 hours later using FACS analysis. The result is graphically depicted in FIG. 3.

### EXAMPLE 2

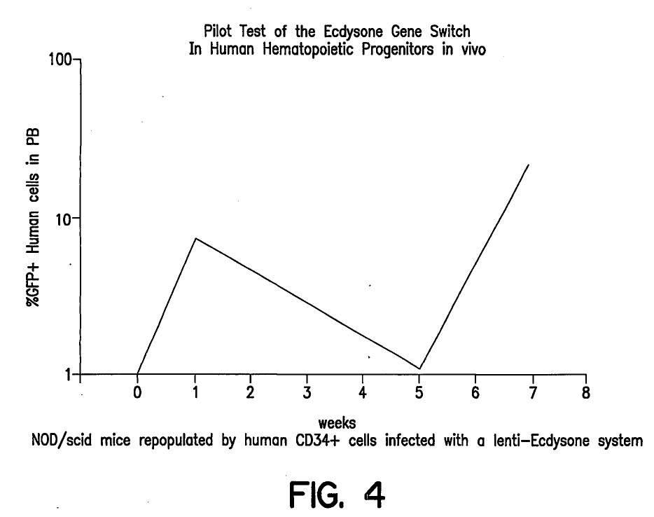

Referring to FIGS. 1 and 5, human hematopoietic stem cells (CD34+) were solated from cord blood and co-infected in vitro with a transactivating lentivector (PG-cPPT-CVgRXR-WPRE) and a reporter lentivector (PG-cPPT-E/GGFP-WPRE) in which the GFP gene is under the control of an ecdysone-responsive promoter. Infected cells were introduced through the tail vein into sub-lethally-irradiated NOD/scid mice. Mice were undisturbed for 6 weeks to allow the bone marrow to reconstitute. They were then bled to check the levels of blood cell repopulation (CD45 positive human cells derived from the transplanted infected progenitors) and the levels of GFP expression in the absence of inducer. Mice were then treated with inducer (ponA) three days in a row (arrows) and subsequently bled to assess the number of GFP positive cells in peripheral blood (PB). After 4 weeks the mice were bled prior to a second round of treatment with inducer. Referring to FIG. 4, the percentage of GFP positive cells in a representative mouse at the various timepoints is shown on the Y axis. This experiment demonstrates the utility of the lentivector-introduced ecdysone-regulated system (its ability to regulate the expression of a reporter gene in a pulsatile manner) for ex vivo/in vivo gene therapy applications and animal model development.

While the invention has been described in detail with reference to certain preferred embodiments thereof, it will be understood that modifications and variations are within the spirit and scope of that which is described.

1. An inducible gene transfer vector, said vector comprising:
a self-inactivating lentiviral 5′ LTR,
a modulator-responsive promoter, wherein said promoter is operatively associated with a gene of interest,
a nuclear import signal,
a promoter operatively associated with nucleic acid encoding a modulator-responsive receptor, and optionally, a silent partner therefor,
an RNA stabilization element, and
a self-inactivating lentiviral 3′ LTR.

2. A vector according to claim 1 wherein said modulator-responsive promoter is responsive to hormone or hormone-like compounds.

3. A vector according to claim 1 wherein said modulator-responsive promoter is responsive to ecdysteroid(s), diacyl hydrazine(s), xenobiotic(s), antibiotics, herbal extracts, or prescription drugs.

4. A vector according to claim 1 wherein said modulator-responsive promoter comprises a binding element for a transcription factor.

5. A vector according to claim 4 wherein said binding element is a hormone response element comprising a direct or inverted repeat motif based on the half site RGBNNM,
wherein:
R is selected from A or G;
B is selected from G, C, or T;
each N is independently selected from A, T, C, or G; and
M is selected from A or C;
with the proviso that at least 4 nucleotides of said —RGBNNM-sequence are identical with the nucleotides at corresponding positions of the sequence AGTTCA, and
wherein said half sites are separated by in the range of 0 up to 15 nucleotides.

6. A vector according to claim 5 wherein said half sites are separated by in the range of 2 up to 6 nucleotides.

7. A vector according to claim 4 wherein said binding element is a GAL4 response element or a tet binding element.

8. A vector according to claim 1 wherein said gene of interest is a therapeutic gene, a reporter gene, a marker gene, an antisense gene, any nucleotide sequence which imparts measurable properties on infected cells containing same, or said gene of interest encodes a therapeutic protein, a reporter protein, a marker protein, a toxic protein, a regulatory protein, an enzyme, a ribozyme, or any amino acid sequence which imparts measurable properties on infected cells containing same.

9. (Cancelled)

10. A vector according to claim 1 wherein said nuclear import signal comprises central polypurine tract and termination sequences of Poll (cPPT).

11. A vector according to claim 1 wherein said promoter operatively associated with nucleic acid encoding a modulator-responsive receptor is constitutively active.

12. A vector according to claim 11 wherein said constitutively active promoter is a viral promoter, a cellular promoter or a tissue specific promoter.

13. A vector according to claim 1 wherein said promoter operatively associated with nucleic acid encoding a modulator-responsive receptor is inducible.

14. A vector according to claim 13 wherein said inducible promoter is a viral promoter or a cellular promoter.

15. A vector according to claim 1 wherein said modulator-responsive receptor comprises a DNA binding domain and a ligand binding domain from a member of the nuclear receptor superfamily.

16. A vector according to claim 15 wherein said member of the nuclear receptor superfamily is a benzoate X receptor (BXR), a constitutively active receptor (CAR), an ecdysone receptor (EcR), an estrogen receptor (ER), a glucocorticoid receptor (GR), a peroxisome proliferator activated receptor (PPAR), a progesterone receptor (PR), a pregnane X receptor (PXR), a retinoic acid receptor (RAR), a retinoid X receptor (RXR), a steroid xenobiotic receptor (SXR), a thyroid hormone receptor (TR) or a vitamin D receptor (VDR).

17. A vector according to claim 1 wherein said silent partner is present.

18. A vector according to claim 17 wherein said silent partner is RXR, usp or functional fragment thereof.

19. (Cancelled)

20. A vector according to claim 17 wherein said nucleic acid encoding said modulator-responsive receptor and said silent partner therefor contains an internal ribosomal entry site.

21. A vector according to claim 1 wherein said RNA stabilization element is a post-transcriptional regulatory element of Woodchuck hepatitis virus (wpre).

22. A gene therapy method for treating a subject, said method comprising exposing said subject to an effective amount of at least one modulator for said modulator-responsive receptor, wherein said subject has previously been treated with a vector according to claim 1.

23-26. (Cancelled)

27. A transactivating lentivector, said lentivector comprising:
a self-inactivating lentiviral 5′ LTR,
a nuclear import signal,
a promoter operatively associated with nucleic acid encoding a modulator-responsive receptor, and optionally, a silent partner therefor,
an RNA stabilization element, and
a self-inactivating lentiviral 3′ LTR.

28. A response lentivector, said lentivector comprising:
a self-inactivating lentiviral 5′ LTR,
a modulator-responsive promoter, wherein said promoter is operatively associated with a gene of interest,
a nuclear import signal,
an RNA stabilization element, and
a self-inactivating lentiviral 3′ LTR.

29. An inducible expression system comprising a transactivating lentivector according to claim 27 and a response lentivector,
wherein said response lentivector comprises:
a self-inactivating lentiviral 5′ LTR,
a modulator-responsive promoter, wherein said promoter is operatively associated with a gene of interest,
a nuclear import signal,
an RNA stabilization element, and
a self-inactivating lentiviral 3′ LTR.

30. A gene therapy method for treating a subject, said method comprising exposing said subject to an effective amount of at least one modulator for a modulator-responsive receptor, wherein said subject has previously been treated with an expression system according to claim 29.

31-34. (Cancelled)

35. A method of imparting to a target cell the ability to inducibly express a gene of interest, said method comprising introducing a gene transfer vector according to claim 1 into said target cells.

36. A cell containing a gene transfer vector according to claim 1.

37. A method for inducibly expressing a gene of interest in a target cell, said method comprising exposing said target cell to a modulator of modulator-responsive receptor, wherein said target cell has been infected with a gene transfer vector according to claim 1.

38-39. (Cancelled)

40. A method of imparting to a target cell the ability to inducibly express a gene of interest, said method comprising introducing an expression system according to claim 29 into said target cells.

41. A cell containing an expression system according to claim 29.

42. A method for inducibly expressing a gene of interest in a target cell, said method comprising exposing said target cell to a modulator of modulator-responsive receptor, wherein said target cell has been infected with an expression system according to claim 29.

43-47. (Cancelled)

48. An ex vivo gene therapy method for treating a subject, said method comprising re-introducing modified cells to a subject, wherein said cells are compatible with said subject, and wherein an inducible gene transfer vector according to claim 1 has been introduced into said cells.

49-51. (Cancelled)

52. An ex vivo gene therapy method for treating a subject, said method comprising re-introducing modified cells to a subject, wherein said cells are compatible with said subject, and wherein said an inducible expression system according to claim 29 has been introduced into said cells.

53-60. (Cancelled)
